(19) United States
(12) Patent Application Publication
(10) Pub. No.: US 2025/0195247 A1
(43) Pub. Date: Jun. 19, 2025

(54) CRIMPER FOR PROSTHETIC HEART VALVES

(71) Applicant: The Regents of the University of Colorado, a Body Corporate, Denver, CO (US)

(72) Inventors: Nicholas Jacobson, Denver, CO (US); Gareth Morgan, Aurora, CO (US); Jenny Elizabeth Zablah Alabi, Englewood, CO (US); Robert Johnathan Elliott, Aurora, CO (US)

(21) Appl. No.: 19/072,539

(22) Filed: Mar. 6, 2025

Related U.S. Application Data
(63) Continuation of application No. PCT/US23/31994, filed on Sep. 5, 2023.
(60) Provisional application No. 63/404,021, filed on Sep. 6, 2022.

(51) Int. Cl. A61F 2/95

(52) U.S. Cl. CPC A61F 2/9524

(57) ABSTRACT

An apparatus and method of crimping at least one stent to a predetermined size. The crimping apparatus may comprise at least one handle, such that at least one user may apply a force to the handle to crimp the perimeter of a stent. Additionally, the crimping apparatus may comprise a safety system configured to inhibit further crimping past a predetermined size. Accordingly, the safety system may comprise at least one stopper, such that once the at least one user selected a predetermined size, via a dial of the safety system, at least one crimping member of the crimping apparatus is prevented from movement further than the predetermined size. Furthermore, the crimping apparatus may comprise a chamfered opening, such that the integrity and structure of the s stent may be maintained during the crimping process.

### CROSS-REFERENCE TO RELATED APPLICATIONS

This nonprovisional application claims priority to PCT application PCTUS2331994 entitled “CRIMPER FOR PROSTHETIC HEART VALVES,” filed Sep. 5, 2023 by the same inventors, which claims priority to provisional application No. 63/404,021, entitled “CRIMPER FOR PROSTHETIC HEART VALVES,” filed Sep. 6, 2022 by the same inventors.

### BACKGROUND OF THE INVENTION

### 1. Field of the Invention

This invention relates, generally, to prosthetic valves. More specifically, it relates to an apparatus and method for optimizing crimping of at least one stented prosthetic to a predetermined size for a surgical procedure.

### 2. Brief Description of the Prior Art

Through the innovation of minimally invasive surgical techniques, such as endovascular grafting, angioplasty, and arthroscopic surgery, surgeons are able to clear blockages, insert valves, remove kidney stones, etc., without requiring substantial surgery. One of the most common biotechnologies associated with these minimally invasive surgical techniques is stents. A stent is generally a cylindrical prosthesis introduced into the lumen of a body vessel via a catheterization technique, primarily used for endovascular procedures. As such, depending on the surgical procedure, the stent may be self-expanding or balloon expandable. For balloon-expandable stents, these stents must be constrained (i.e., “crimped”) from an initial large diameter to a smaller diameter prior to advancement to a treatment site in the body. Before crimping, a balloon expandable stent is typically placed over an expandable balloon on a catheter shaft. In cases where the stent was manufactured in its fully crimped diameter, the stent is first, expanded by the manufacturer and then finally crimped on the balloon.

As previously stated, currently known crimping techniques generally rely on “pre-crimped” stents from manufacturers to use for implementation. The use of “pre-crimped” stents, however, comes with substantial drawbacks. For example, a typical coronary stent diameter is only 1.5 to 4.0 mm in its expanded size, while a stented prosthetic valve diameter will typically be in the range of about 19 to 29 mm, which is at least 5 times as large as a coronary stent. Furthermore, coronary stents are primarily stand-alone devices while, in prosthetic valves, the stent functions as a scaffold to hold the valve structure in the predetermined location. As such, after deployment, it is often desirable to preserve such valves in the open (i.e., expanded) diameter inside a preserving solution. Therefore, using this procedure, it may be necessary to crimp the valve in the operation room a few minutes before implantation, therefore precluding pre-crimping by the manufacturer over a balloon.

Recent advances in crimping technology have enabled use of crimping devices within a sterile field, including in the operating room (hereinafter “OR”). As the stent is placed around a predetermined balloon size, at least one jaw of the device crimps the stent and valve around the predetermined balloon size. However, currently known crimping apparatuses using this technology-such as the one disclosed in U.S. Pat. No. 7,895,876 (issued Mar. 1, 2011)-require that the surgeon, technician, or any user performing the crimping on the stent to estimate, by hand, an exact diameter needed for the stent. Accordingly, applications of such crimping apparatuses are inefficient as they introduce substantial human error into the crimping processes. For example, once a stent has been crimped, it is exceedingly difficult to expand and re-crimp the stent without comprising the integrity of the stent. As such, using a re-crimped stent may put the patient in substantial and/or life-threating danger. Furthermore, by estimating by hand, it is likely that multiple stents may be incorrectly crimped, requiring additional stents to be available on hand so that the user may reattempt to correctly crimp the stent, substantially increasing cost of the operation. Thus, currently known crimping apparatuses are incapable of crimping any stent to its required size, without introducing the potential of human error.

Accordingly, what is needed is safe, efficient, and easy-to-use apparatus and method for optimized crimping of at least one stented prosthetic valve for surgical insertion. However, in view of the art considered as a whole at the time the present invention was made, it was not obvious to those of ordinary skill in the field of this invention how the shortcomings of the prior art could be overcome.

### SUMMARY OF THE INVENTION

The long-standing but heretofore unfulfilled need, stated above, is now met by a novel and non-obvious invention disclosed and claimed herein. In an aspect, the present disclosure pertains to apparatus and method for optimizing crimping of at least one stented prosthetic to a predetermined size for a surgical procedure.

In an aspect, the present invention includes a crimping apparatus for crimping a stent to a predetermined size. The crimping apparatus includes a casing having an opening for receiving the stent. The opening in the casing may have a chamfered entrance.

The crimping apparatus further includes a handle rotatably mounted within the casing. The handle includes a plurality of stopper holes. The plurality of stopper holes in the handle are collectively arranged in an arcuate shape and may partially form a Fibonacci spiral. Each stopper hole also includes a sloped entrance section, thereby allowing the handle to be rotated back to the starting position.

The crimping apparatus further includes a radial housing mounted within the casing in a non-rotatable manner. At least one crimping member is in translatable communication with the radial housing and configured to reduce an effective diameter of the opening in the casing when the handle is rotated.

The device also includes a safety system. The safety system includes a safety stopper and an external dial. The external dial has a plurality of predetermined stages, wherein each stage corresponds to a particular location of the safety stopper. The external dial can also include indicia for each of the predetermined stages. The indicia indicate an expected crimped diameter when the external dial of the safety system is set to one of the predetermined stages.

The safety stopper is configured to engage a predetermined stopper hole when the external dial is located at one of the plurality of predetermined stages and the handle is rotated a predetermined distance from a starting position, such that the handle is prevented from rotating further. The safety stopper includes a spring-loaded plunger and may extend through an arcuate track in the casing.

The safety device can further include a cylindrical extension projecting outwardly from the casing. A plurality of stage holes is circumferentially spaced about the cylindrical extension. In addition, the safety system has a stage stopper. The stage stopper may include a spring-loaded plunger and is configured to engage one of the plurality of stage holes when the stage stopper is aligned with one of the plurality of stage holes. Furthermore, the external dial location determines a location of the stage stopper relative to the plurality of stage holes.

The present invention further includes a method of crimping a stent. The method includes acquiring a crimping apparatus for crimping a stent to a predetermined size. The crimping apparatus includes a casing having an opening for receiving the stent; a handle, having a plurality of stopper holes, rotatably mounted within the casing; at least one crimping member configured to reduce a diameter of the opening; and a safety system having a safety stopper and an external dial having a plurality of predetermined stages corresponding to a particular location of the safety stopper. The safety stopper is configured to engage a predetermined stopper hole when the external dial is located at one of the plurality of predetermined stages and the handle is rotated a predetermined distance from a starting position, such that the handle is prevented from rotating further. The acquired stent may have any of the other features described above and herein.

The method of the present invention further includes inserting the stent into the opening, setting the external dial to one of the plurality of predetermined stages corresponding to an expected crimped diameter, rotating the handle from the starting position until the safety stopper engages one of the predetermined stopper holes, and rotating the handle back to the starting position after the handle stops rotating as a result of the safety stopper engaging one of the predetermined stopper holes.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not restrictive.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the invention, reference should be made to the following detailed description, taken in connection with the accompanying drawings, in which:

FIG. 1A is a top-down perspective view of a crimping apparatus, according to an embodiment of the present disclosure.

FIG. 1B is an exploded view of a crimping apparatus, according to an embodiment of the present disclosure.

FIG. 2 is an internal perspective view of a first body of a casing of a crimping apparatus, according to an embodiment of the present disclosure.

FIG. 3A is an external perspective view of a second body of a handle of a crimping apparatus, according to an embodiment of the present disclosure.

FIG. 3B is an external perspective view of a second body of a handle of a crimping apparatus, according to an embodiment of the present disclosure.

FIG. 4 is a perspective view of a handle of a crimping apparatus comprising the first body of FIG. 2 and the second body of FIGS. 3, according to an embodiment of the present disclosure.

FIG. 5 is a front perspective view of a radial housing, according to an embodiment of the present disclosure.

FIG. 6 is a rear perspective view of a radial housing, according to an embodiment of the present disclosure.

FIG. 7 is a front perspective view of a front portion of a casing of a crimping apparatus, according to an embodiment of the present disclosure.

FIG. 8 is a rear perspective view of a front portion of a casing of a crimping apparatus, according to an embodiment of the present disclosure.

FIG. 9 is a perspective view of a rear portion of a casing of a crimping apparatus, according to an embodiment of the present disclosure.

FIG. 10 is a side view of the rear portion of the casing of FIG. 9, according to an embodiment of the present disclosure.

FIG. 11 is a perspective view of a casing comprising the front portion of FIG. 8 and the rear portion of FIG. 9, according to an embodiment of the present disclosure.

FIG. 12 is a perspective view of a stopper of a crimping apparatus, according to an embodiment of the present disclosure.

FIG. 13A is a perspective view of a safety system of a crimping apparatus, according to an embodiment of the present disclosure.

FIG. 13B is a front view showing the crimping device with the front portion of the casing removed and the safety system shown in a transparent manner to depict how the internal components of the safety system interact with the handle.

FIGS. 14A-14C are perspective views of the safety system of FIG. 13, according to an embodiment of the present disclosure. FIG. 14A is a side view of the safety system of FIG. 13A. FIG. 14B is a top-down view of the safety system of FIG. 13. FIG. 14C is a rear view of the safety system of FIG. 13.

FIG. 15A is a perspective view of a crimping member of a crimping apparatus, according to an embodiment of the present disclosure.

FIG. 15B is a side view of a crimping member of a crimping apparatus, according to an embodiment of the present disclosure.

FIG. 15C is a rear view of a crimping member of a crimping apparatus, according to an embodiment of the present disclosure.

FIG. 16 is a perspective view of a radial housing temporarily affixed to a front portion of a casing, according to an embodiment of the present disclosure.

FIG. 17 is a perspective view of at least one crimping member temporarily affixed to a radial housing, according to an embodiment of the present disclosure.

FIG. 18 is a perspective view of at least one crimping member temporarily affixed to a radial housing, where the radial housing is temporarily affixed to a front portion and a rear portion of a casing of a crimping apparatus, according to an embodiment of the present disclosure.

FIG. 19 is a perspective view of a radial housing temporarily affixed to a rear portion of a casing of a crimping apparatus, according to an embodiment of the present disclosure.

FIG. 20 is a perspective view of a crimping apparatus and a safety system of the crimping apparatus disengaged, according to an embodiment of the present disclosure.

FIG. 21 is a perspective view of a crimping apparatus engaged and a safety system of the crimping apparatus disengaged, according to an embodiment of the present disclosure.

FIG. 22 is a perspective view of a crimping apparatus engaged and a safety system of the crimping apparatus engaged, according to an embodiment of the present disclosure.

FIG. 23 is a flow chart depicting the steps of a method of crimping a stent to a predetermined size using a crimping apparatus, according to an embodiment of the present disclosure.

### DETAILED DESCRIPTION OF THE INVENTION

In the following detailed description of the preferred embodiments, reference is made to the accompanying drawings, which form a part thereof, and within which are shown by way of illustration specific embodiments by which the invention may be practiced. It is to be understood that one skilled in the art will recognize that other embodiments may be utilized, and it will be apparent to one skilled in the art that structural changes may be made without departing from the scope of the invention. Elements/components shown in diagrams are illustrative of exemplary embodiments of the disclosure and are meant to avoid obscuring the disclosure. Any headings used herein are for organizational purposes only and shall not be used to limit the scope of the description or the claims. Furthermore, the use of certain terms in various places in the specification, described herein, are for illustration and should not be construed as limiting.

### Definitions

Reference in the specification to “one embodiment,” “preferred embodiment,” “an embodiment,” or “embodiments” means that a particular feature, structure, characteristic, or function described in connection with the embodiment is included in at least one embodiment of the disclosure and may be in more than one embodiment. The appearances of the phrases “in one embodiment,” “in an embodiment,” “in embodiments,” “in alternative embodiments,” “in an alternative embodiment,” or “in some embodiments” in various places in the specification are not necessarily all referring to the same embodiment or embodiments. The terms “include,” “including,” “comprise,” and “comprising” shall be understood to be open terms and any lists that follow are examples and not meant to be limited to the listed items.

As used in this specification and the appended claims, the singular forms “a,” “an,” and “the” include plural referents unless the content clearly dictates otherwise. As used in this specification and the appended claims, the term “or” is generally employed in its sense including “and/or” unless the context clearly dictates otherwise.

As used herein, “about” means approximately or nearly and in the context of a numerical value or range set forth means±15% of the numerical.

As used herein, the term “fastening mechanism” refers to any device known in the art which may temporarily connect at least two devices together. The fastening mechanism may be a screw, a nail, a magnet, and/or a rivet. For ease of reference, the exemplary embodiment, described herein, refers to a screw, but this description should not be interpreted as exclusionary of other fastening devices.

As used herein, the term “restrictions” refers to any aspect known in the art which may prevent smooth translation of a device. For ease of reference, the exemplary embodiment, described herein, refers to increased friction between at least two surfaces, but this description should not be interpreted as exclusionary of other restrictions.

All numerical designations, including ranges, are approximations which are varied up or down by increments of 1.0, 0.1, 0.01 or 0.001 as appropriate. It is to be understood, even if it is not always explicitly stated, that all numerical designations are preceded by the term “about”. It is also to be understood, even if it is not always explicitly stated, that the compounds and structures described herein are merely exemplary and that equivalents of such are known in the art and can be substituted for the compounds and structures explicitly stated herein.

Wherever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Wherever the term “no more than,” “less than,” or “less than or equal to” precedes the first numerical value in a series of two or more numerical values, the term “no more than,” “less than” or “less than or equal to” applies to each of the numerical values in that series of numerical values. For example, less than or equal to 1, 2, or 3 is equivalent to less than or equal to 1, less than or equal to 2, or less than or equal to 3.

### Crimping Apparatus:

The present disclosure pertains to a crimping apparatus denoted by reference numeral 100 and method of use denoted by reference numeral 200. Crimping apparatus 100 is configured to crimp at least one stent 174 to a predetermined size based on a required procedure for a patient. In an embodiment, stent 174 may comprise a coronary stent, a drug eluting stent, bioresorbable vascular scaffold stent, ridaformolimus-eluting stent, zotarolimus-eluting stent a bar-metal stent, and/or any stent known in the art which may be used during a surgical procedure. Additionally, stent 174 may comprise at least one valve, such that it may regulate the flow of fluids and/or blood through the body of the patient.

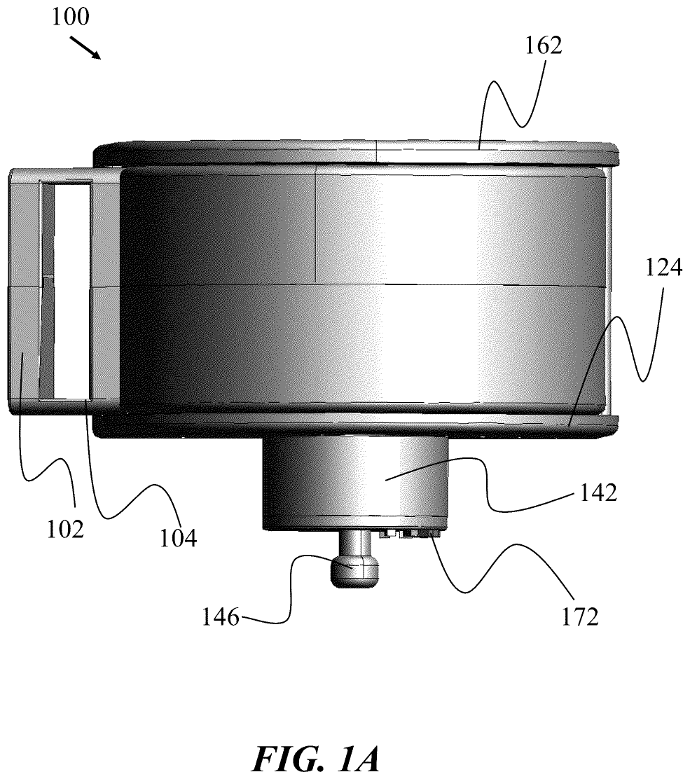

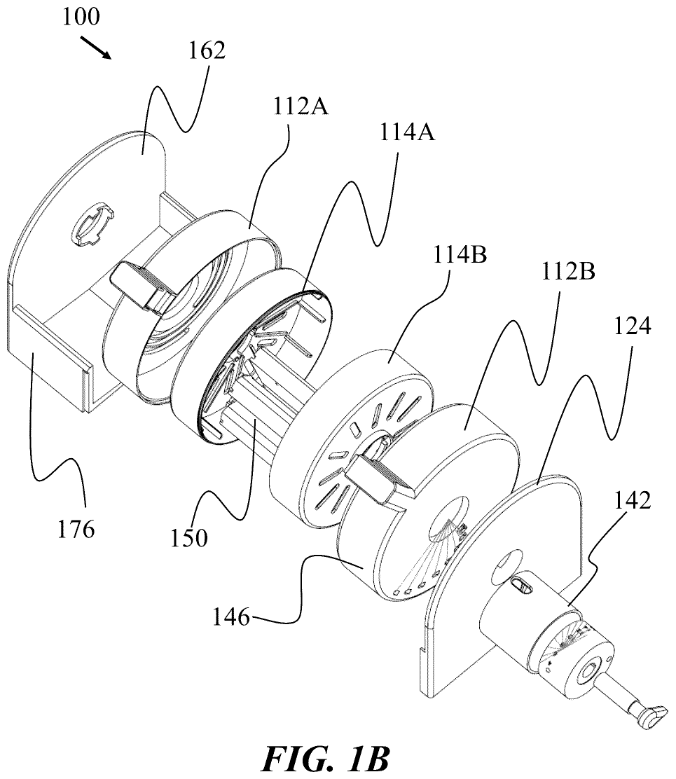

As shown in FIGS. 1-4, crimping apparatus 100 includes casing 176 comprising front portion 124 and rear portion 162. Crimping apparatus 100 also includes handle 112 and radial housing 114 residing within casing 176. Handle 112 may be rotatably affixed to front portion 124 and/or rear portion 162 of casing 176 while radial housing 114 is non-rotatably fixed to front portion 124 and/or rear portion 162 of casing 176. Radial housing 114 includes a plurality of slots 116 configured to direct crimping members 150 in a radial direction to crimp stent 174.

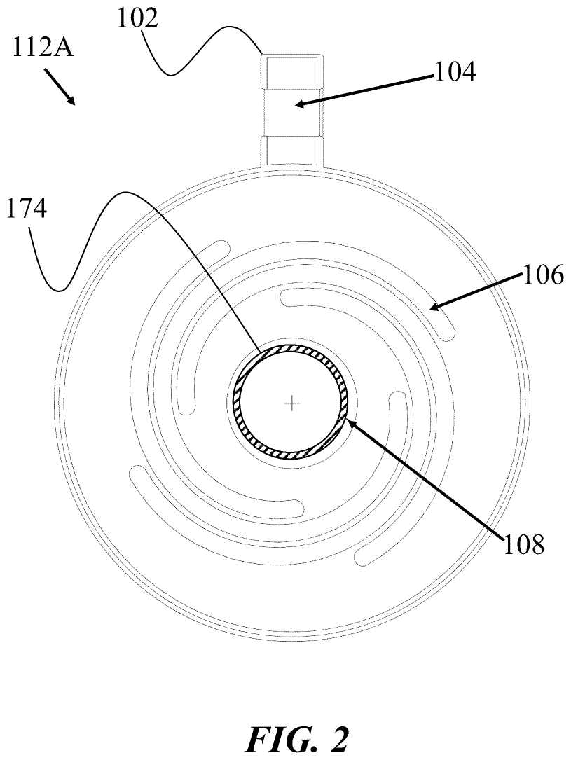

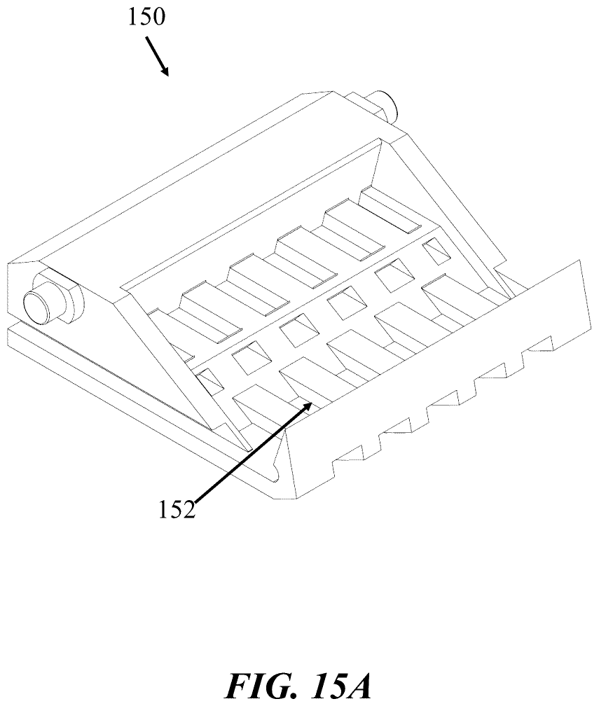

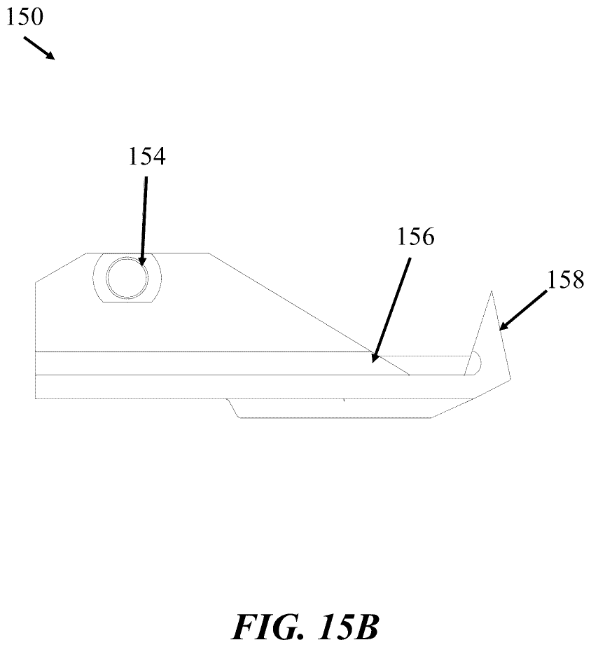

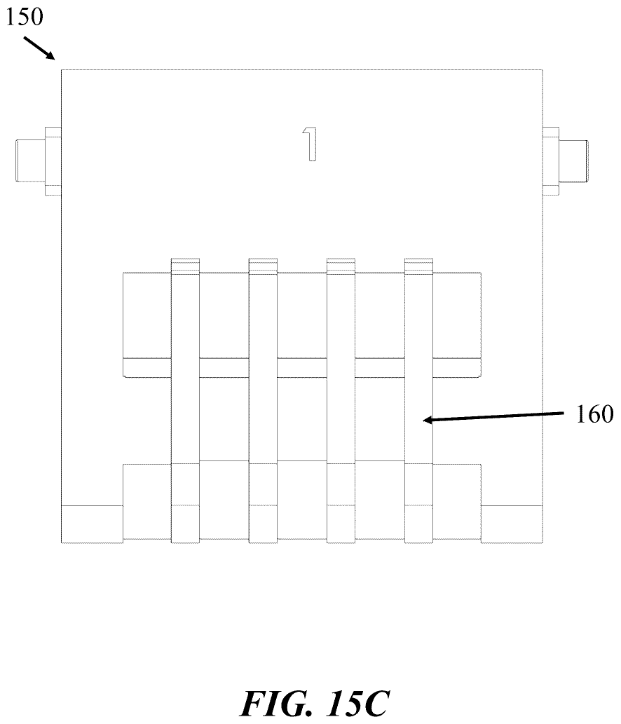

As shown in FIG. 2, handle 112 includes a plurality of generally circumferential, spiral grooves 106 disposed within first wall 112A of handle 112. The plurality of grooves 106 are interconnected with crimping members 150, as shown in FIGS. 15A-15C, such that rotation of handle 112 causes translation of crimping members 150. More specifically, as handle 112 rotates, crimping members 150 are directed in a radial direction about slots 116 in radial housing 114 to crimp stent 174. Additionally, as shown in FIG. 2, handle 112 includes opening 108, such that stent 174 may be disposed within first opening 108 during crimping.

Handle 112 also includes interface 102, such that at least one user may interact with crimping apparatus 100. In this manner, as shown in FIGS. 1-2 and 4, at least one interface 102 may comprise at least one aperture 104, allowing a user to grip interface 102 when applying force to crimping apparatus 100. In this embodiment, the user can insert his/her fingers through aperture 104, such that the user may grasp interface 102 within a palm of their hand, allowing the user to transfer an optimal amount of force to crimping apparatus 100 without any excess waste and/or exertion.

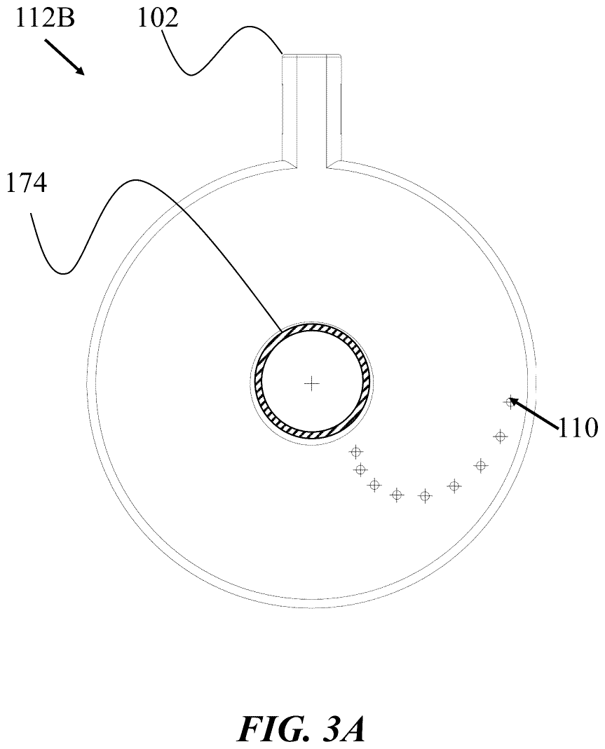

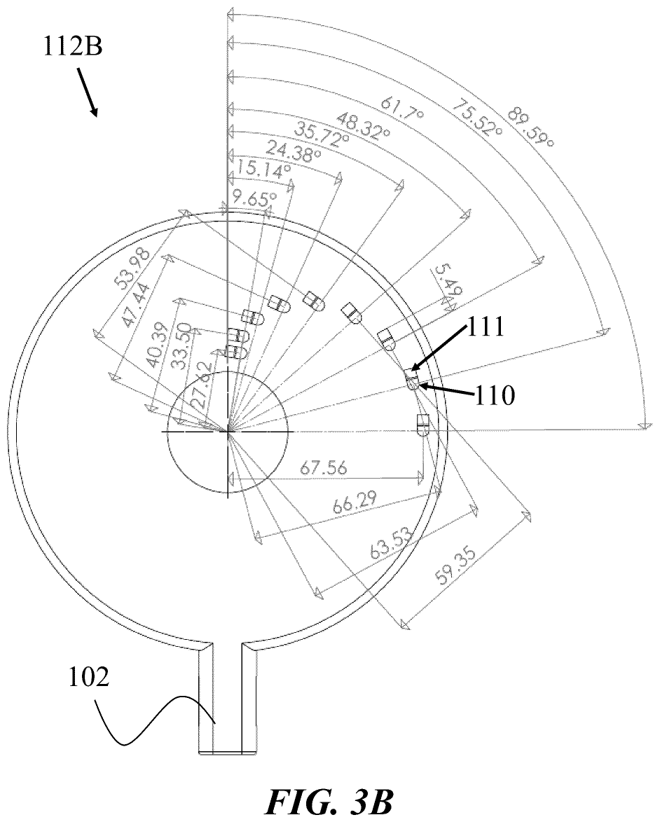

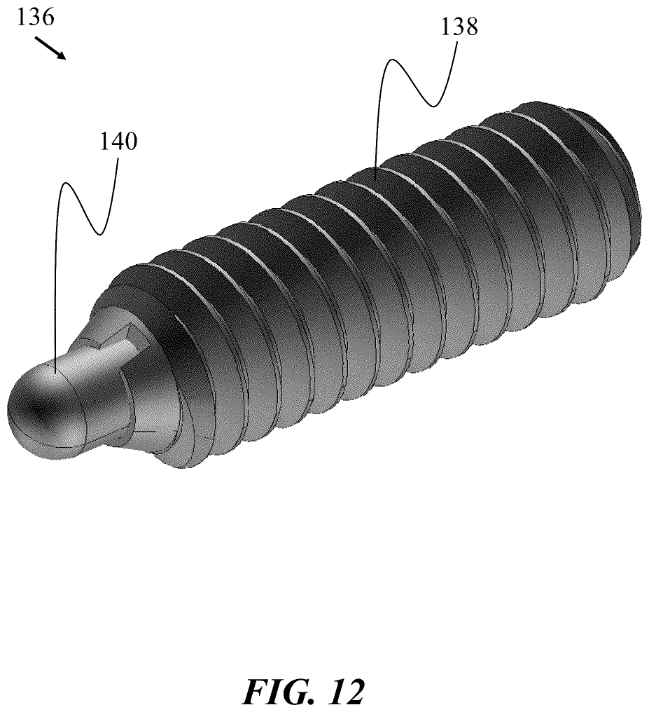

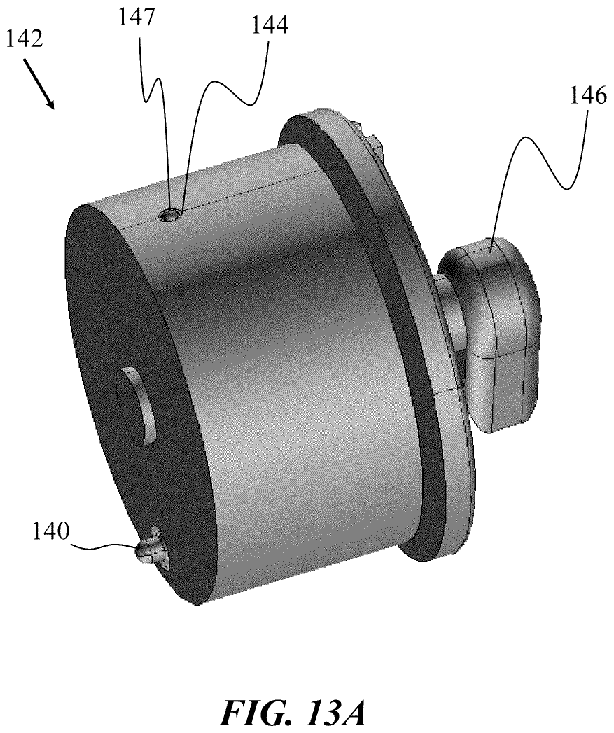

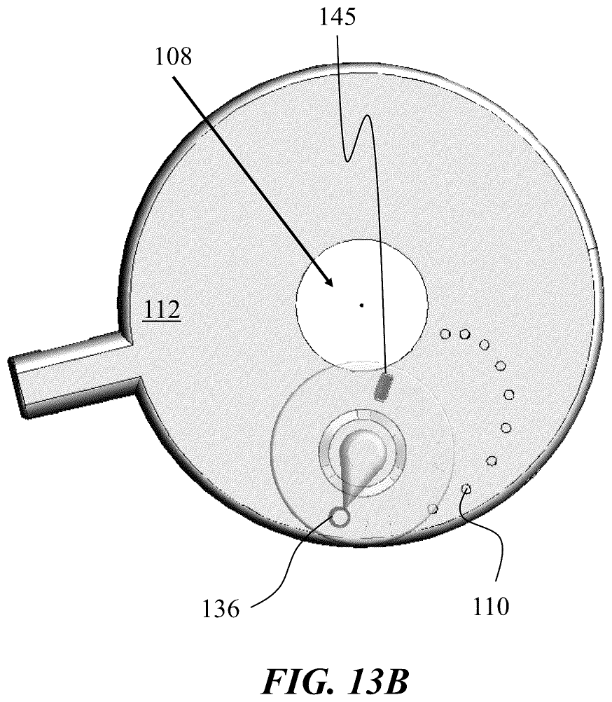

Additionally, as shown in FIG. 3, second wall 112B of handle 112 includes a plurality of stopper holes 110. As will be explained, stopper holes 110 are configured to retain plunger 140 of safety stopper 136 of safety system 142 (see FIGS. 12-13). In this manner, when a required size for stent 174 is determined, safety stopper 136 may be positioned to the appropriate location such that an appropriate stopper hole 110 translates into alignment with safety stopper 136 as handle 112 is rotated, such that the exact size of stent 174 may be obtained.

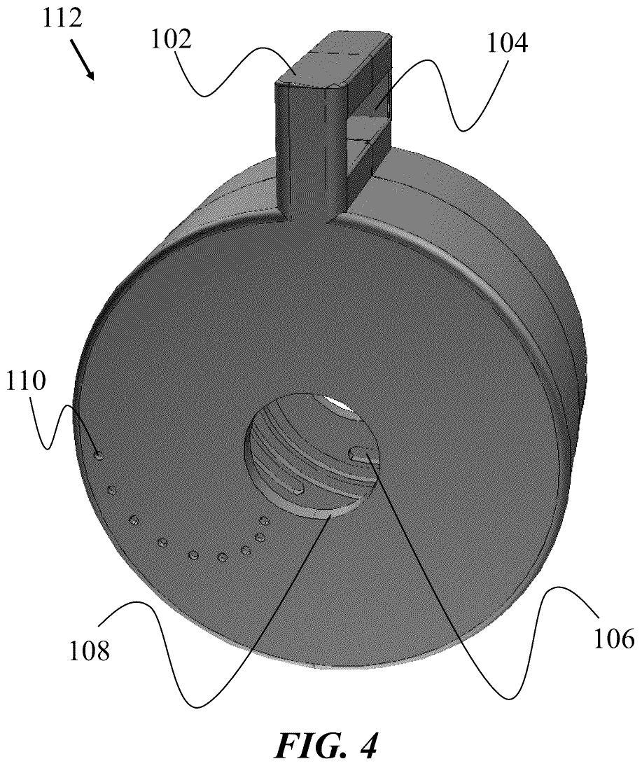

As depicted best in FIGS. 3-4, the collection of the plurality of stopper holes 110 is arranged in an arcuate orientation extending generally in a radial direction. In some embodiments, the plurality of stopper holes 110 are arranged along a Fibonacci spiral. The plurality of stopper holes 110 may further be arranged in accordance with the dimensional and rotational offsets depicted in FIG. 3B. This orientation allows handle 112 to rotate as needed until safety stopper 136 aligns with one of the stopper holes 110 and projects into said hole. As will be explained in greater detail, the plurality of stopper holes 110 in this unique orientation enables a user to easily and precisely adjust the degree of rotation and thus extent of crimping of stent 174.

Each of the stopper holes 110 may further include a sloped entrance section 111 as shown in FIG. 3B. The sloped entrance section 111 allows the spring-loaded, dome-shaped plunger 140 to transition out of each hole 110 when handle 112 is rotated back towards the starting position.

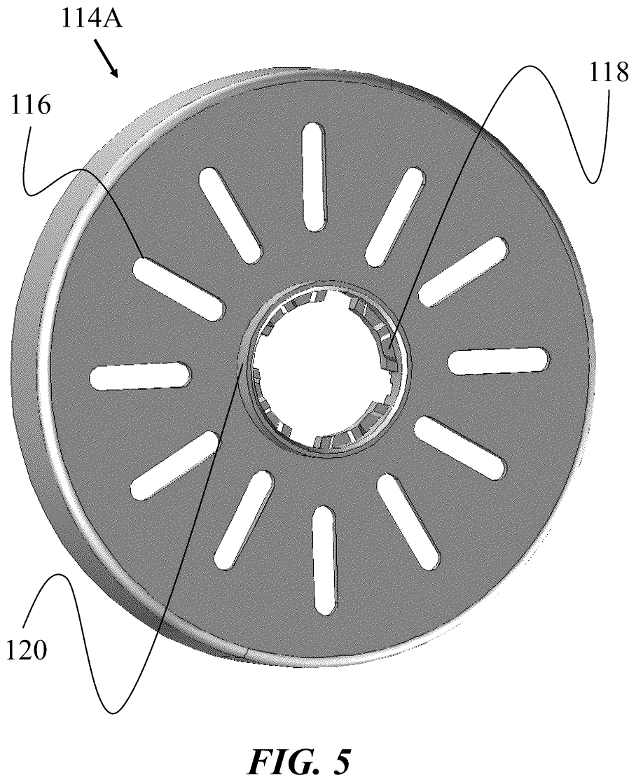

FIG. 5 depicts first wall 114A of radial housing 114 of crimping apparatus 100, according to an embodiment of the present disclosure. It should be noted that second wall 114B of radial housing 114 is of an identical construction as depicted. However, the construction of the first and second walls 114A and 114B may vary in some embodiments.

Each wall 114A and 114B of radial housing 114 includes an opening 120 that is generally in axial alignment with opening 108 in handle 112 to allow for the passage of stent 174. Openings 120 each include at least one protrusion 118. Protrusions 118 are configured to engage front portion 124 or rear portion 162 of casing 176 to prevent rotation of radial housing 114 relative to casing 176. However, radial housing 114 may be configured to be temporarily affixed to handle 112, such that radial housing remains stationary as a handle 112 rotates.

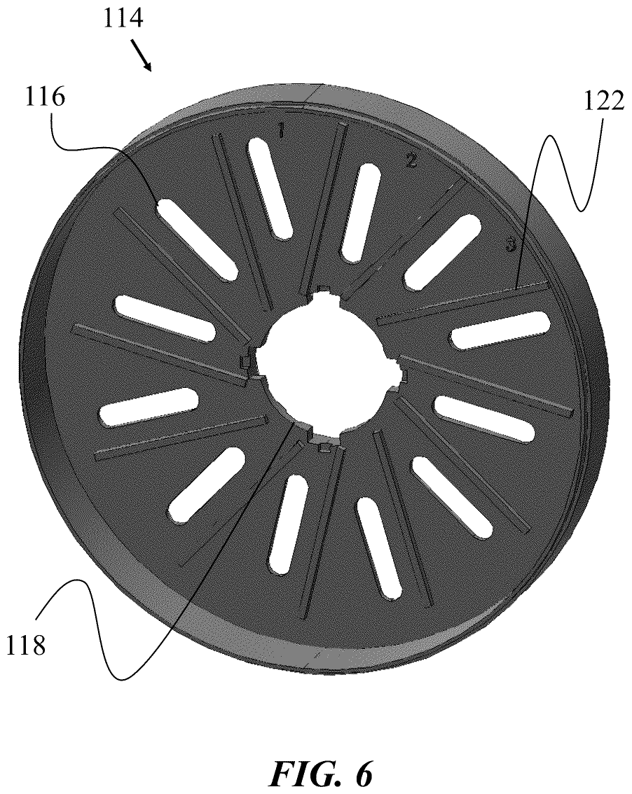

As previously noted, each wall 114A and 114B also comprises a plurality of slots 116. Furthermore, as shown in FIG. 6, in an embodiment, each wall 114A and 114B may comprise a plurality of barriers 122 on an interior surface. As such, in this embodiment, each slot 116 may be separated by at least one barrier 122, such that each slot 116 is separated by an equal distance. In some embodiments, at least one slot 116 may be separated from at least one alternative slot 116 at a variable distance.

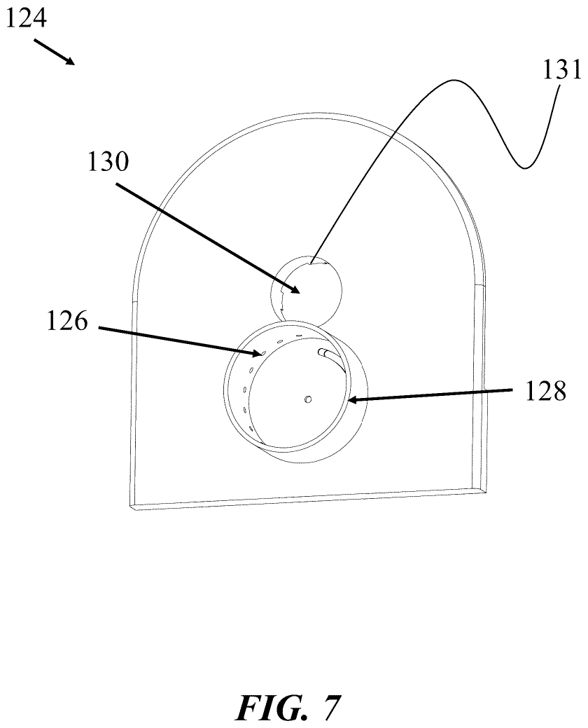

As previously stated, and shown in FIGS. 7-11, in conjunction with FIG. 1, in an embodiment, crimping apparatus 100 may comprise a casing having a front portion 124 and a rear portion 162. Front portion 124 and rear portion 162 may each include openings 130, 166, respectively, such that an aperture extends through crimping apparatus 100 to receive stent 174 and allow for precise crimping of stent 174 to a predetermined size at a predetermined position.

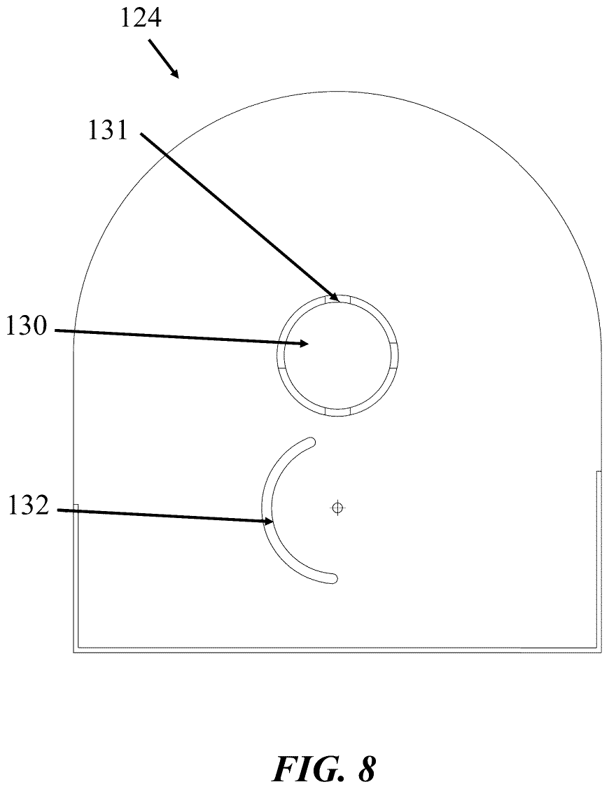

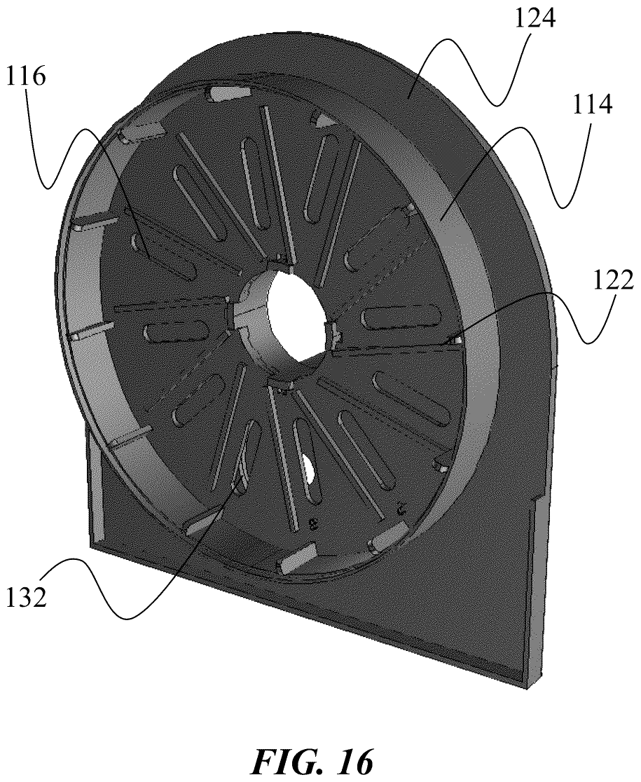

As shown in FIGS. 7-8, opening 130 of front portion 124 may comprise at least one front jut 131. Front jut 131 extends towards second radial wall 114B and has a sufficient extent to pass through opening 108 in handle section 112B. Front jut 131 is configured to have a complimentary shape to the at least one protrusion 118 of second radial wall 114B. In this manner, protrusion(s) 118 operably interconnects with front jut 131, such that second radial wall 114B and front portion 124 of casing 176 are secured to each other in a non-rotatable manner, as shown in FIG. 16, while handle 112 remains free to rotate relative to radial housing 114 and casing 176.

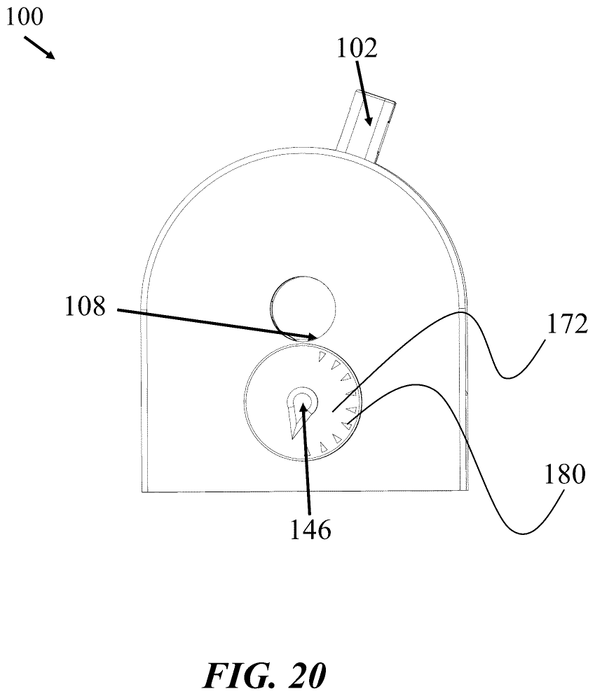

As shown in FIG. 7, front portion 124 further comprises a safety system housing 128. Safety system housing 128 is cylindrical in shape and extends outwardly from front portion 124. Safety system housing 128 is configured to, at least temporarily, couple safety system 142 to front portion 124 of casing 176. Additionally, as shown in FIG. 7, in conjunction with FIGS. 20-22, front portion 124 and/or safety system 142 includes a plurality stage hole 126, 144 respectively. As depicted, the plurality of stage holes 126 reside in safety system housing 128 and a single aperture 144 resides in safety system 142 as shown in FIG. 13. However, the hole locations can be inverted such that the plurality of stage holes 126 reside in safety system 142 and aperture 144 resides in safety system housing 128.

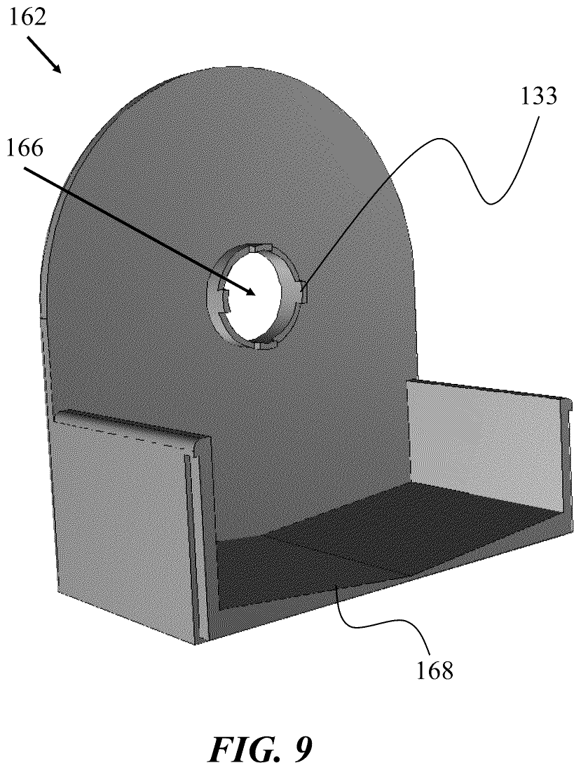

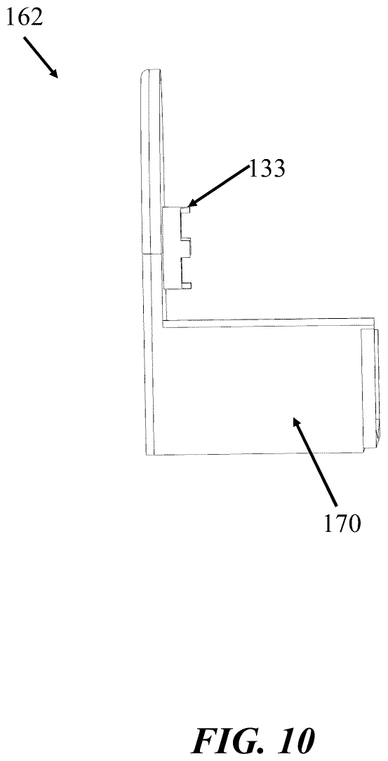

As shown in FIGS. 9-10, rear portion 162 of casing 176 includes at least one rear jut 133. Rear jut 133 extends towards first radial wall 114A and has a sufficient extent to pass through opening 108 in handle section 112A. Rear jut 133 is configured to have a complimentary shape to the at least one protrusion 118 of second radial wall 114A. In this manner, protrusion(s) 118 operably interconnects with rear jut 133 of opening 130, such that second radial wall 114A and rear portion 162 of casing 176 are secured to each other in a non-rotatable manner, as shown in FIG. 16, while handle 112 remains free to rotate relative to radial housing 114 and casing 176.

Moreover, as shown in FIG. 10, in this embodiment, rear portion 162 may comprise a handle stop 170, such that when the at least one user exerts force upon handle 112, it may rotationally translate toward handle stop 170. In this manner, once handle 112 reaches handle stop 170, handle 112 may be prevented from rotationally translating further.

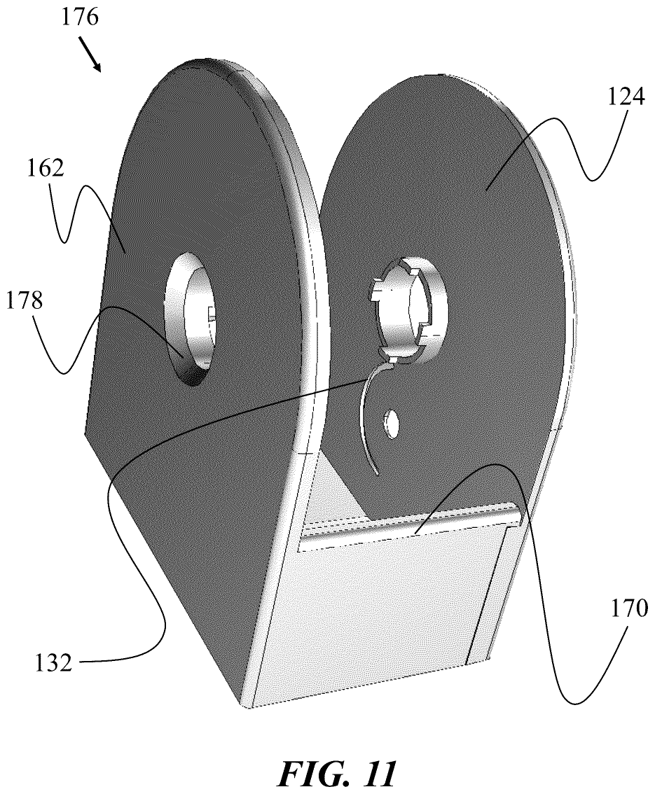

Furthermore, as shown in FIG. 11, opening 166 of rear portion 162 may comprise a chamfered entrance 178, such that the at least one stent 174 may be introduced to crimping apparatus 100 with minimal restrictions. Additionally, in this embodiment, chamfered entrance 178 may be configured to allow at least one stent 174 comprising variable circumference and length to enter crimping apparatus 100.

As previously noted, crimping apparatus 100 further comprises safety system 142. Safety system 142 works in conjunction with front portion 124 to crimp stent 174 to one of a plurality of a predetermined sizes as defined by the stages 180 on dial 172. External knob 146 is configured to be rotated to one of a plurality of stages 180 on dial 172, which conveys to the user exactly how far stent 174 will be crimped. Each stage 180 can be visually identifiable through indicia printed on dial 172. For example, each stage 180 may identify the expected crimped diameter of stent 174 when crimping device 100 is fully crimped at a particular stage.

Referring to FIGS. 12-14, safety system 142 may comprise at least one safety stopper 136, which may be configured to be retained in at least one stopper hole 110 of handle 112 when appropriately aligned. In this manner, at least one safety stopper 136 may comprise a fastening mechanism, such that at least one safety stopper 136 may comprise a proximal body and a distal body. As such, in this embodiment, at least one portion of the distal body of at least one safety stopper 136 may comprise a fastening mechanism 138, such that at least one safety stopper 136 may be configured to be, at least temporarily, affixed to safety system 142.

Additionally, as shown in FIG. 12, in conjunction with FIGS. 3, 4, 10, and 11, and at least one portion of the proximal body of at least one safety stopper 136 may comprise a spring plunger 140. Plunger 140 of safety stopper 136 extends through track 132 in front portion 124. Plunger 140 can be translated through track 132, such that once the at least one stopper 136 is positioned complementary to at least one stopper hole 110, spring plunger 140 engages said hole. As a result of plunger 140 becoming frictionally retained by the at least one stopper hole 110, handle 112 is prevented from rotationally translating past a predetermined distance, based on stage 180 that was selected via knob 146. In some embodiments, spring plunger 140 of at least one safety stopper 136 may be retained via magnets, adhesive, and/or any component known in the art which may be configured to temporarily affix at least two devices together.

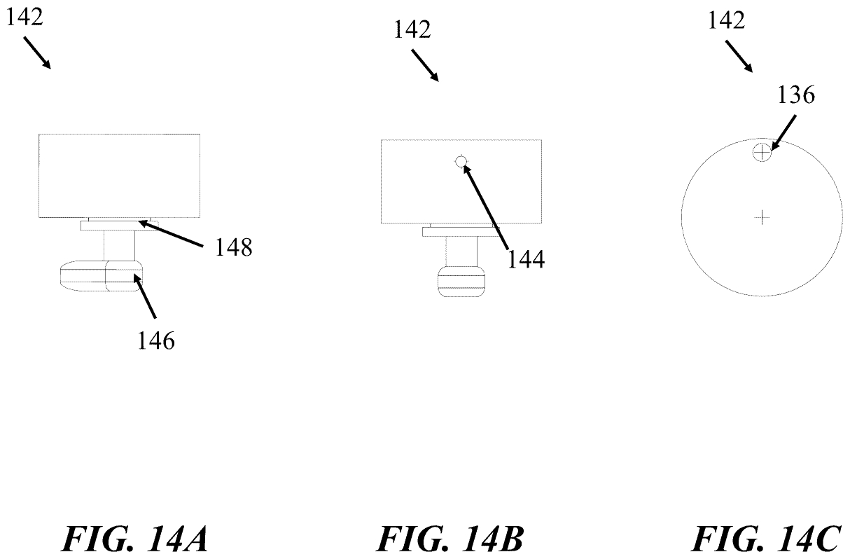

Additionally, in an embodiment, as shown in FIGS. 13-14C, in conjunction with FIG. 7, safety system 142 may comprise at least one aperture 144 and stage stopper 145. Stage stopper 145 also preferably includes a spring plunger 147 and is configured to extend in a radial direction beyond aperture 144 when aligned with one of the stage holes 126. Dial 146 can be rotated, such that stage stopper 145 can align with at least one stage hole 126 allowing plunger 147 to temporarily protrude into at least one stage hole 126 of safety system housing 128 of front portion 124. In this embodiment, when aperture 144 and at least one stage hole 126 are positioned complimentary to each other, spring plunger 147 of stage stopper 145 may be frictionally retained in the stage hole 126 thereby generally preventing accidental rotation of safety system 142 relative to front portion 124 and securing the location of safety stopper 136 at a specific location within track 132. As a result, rotation of handle 112 will, at some predetermined degree of rotation, bring one of stopper holes 110 into alignment with spring plunger 140 of safety stopper 136. Accordingly, in this embodiment, handle 112 may be prevented from performing a full rotation to handle stop 170 as determined by the location of the safety stopper 136 in the path of one of the stopper holes 110. In some embodiments, spring plunger 147 of stage stopper 145 may be retained via magnets, adhesive, and/or any component known in the art which may be configured to temporarily affix at least two devices together.

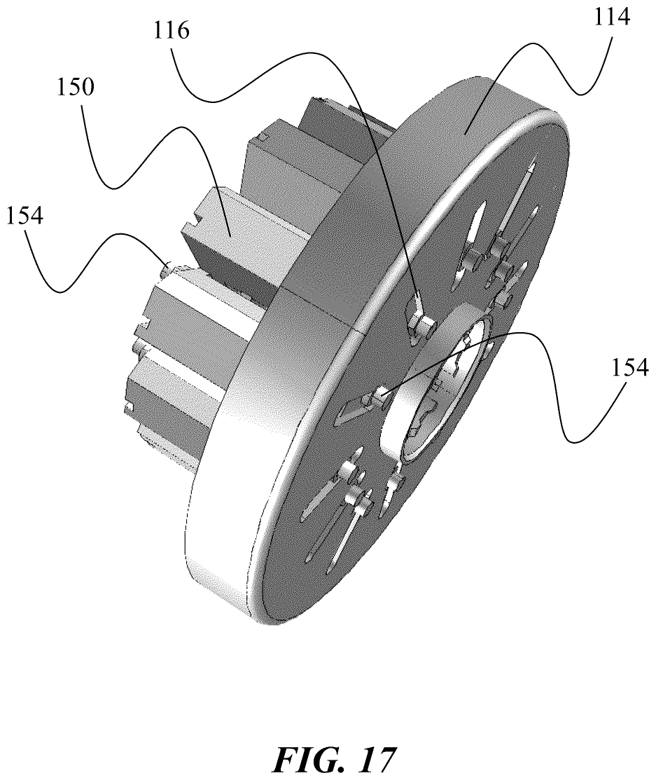

Crimping apparatus 100 further comprises at least one crimping member 150. FIGS. 15A-15C, in conjunction with FIG. 6, depict a crimping member 150 of crimping apparatus 100, according to an embodiment of the present disclosure. The at least one crimping member 150 may be configured to be temporarily affixed to radial housing 114, via at least one barrier 122 of radial housing 114 and crimper slot 156 of at least one crimping member 150. Accordingly, when at least one crimping member 150 is temporarily affixed to radial housing 114, at least one connector 154 may be configured to be disposed within at least one slot 116 of radial housing 114, as shown in FIG. 17.

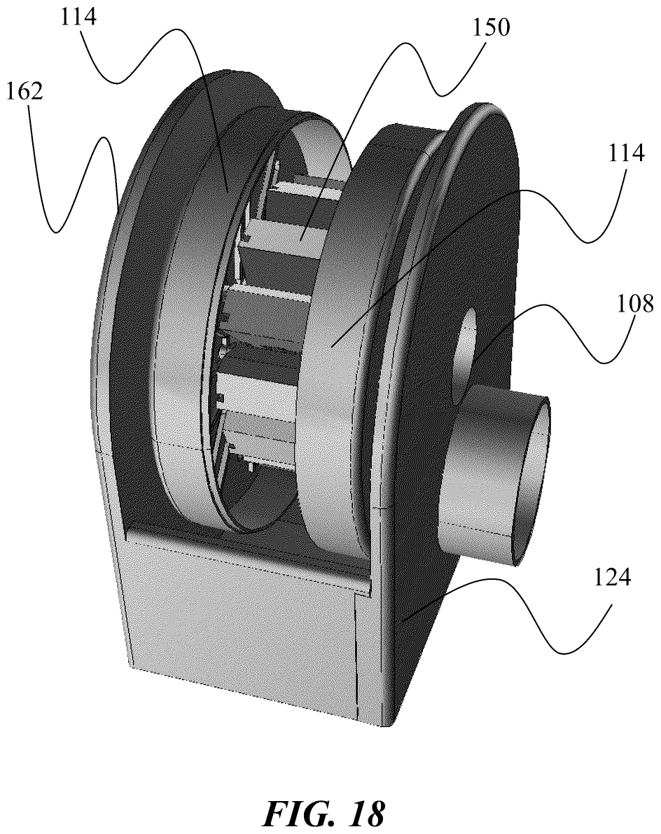

In addition, as shown in FIGS. 15A-15C, in conjunction with FIG. 2 and FIG. 18, the at least one connector 154 of at least one crimping member 150 may be configured to extend through at least one slot 116 of radial housing 114, such that at least a portion of at least one connector 154 may be disposed in at least one groove 106 of handle 112. Because groove 106 spirals inwardly, rotation of handle 112 applies an inwardly radial force on connectors 154 causing connector 154, and in turn crimping members 150, to translate in a radial direction about slots 116 in stationary radial housing 114. As such, when a force is applied to interface 102 of handle 112, at least one crimping member 150 may be configured to proportionally translate, such that the circumference of the at least one aperture extending throughout crimping apparatus 100 is decreased. Accordingly, as the circumference of opening 108 is decreased, each of the crimping members 150 are configured to overlap, allowing a proportion force to be applied to each portion of the at least one stent 174. In this manner, the stent is proportionally crimped based on the predetermined size as required by the procedure.

Furthermore, as the at least stopper 136 and the at least one stopper hole 110 align, based upon the preselected stage 180 of dial 172 of safety system 142, handle 112 is prevented from further rotation. Each of the crimping members 150 will have translated through each of the grooves 106 of handle 112 proportional to the rotation of handle 112. As such, the force, as applied to handle 112, via interface 102, may only be applied to each portion of at least one stent 174, for a predetermined rotation, such that the circumference of the at least one stent 174 is decreased by a predetermined amount, allowing crimping at specific stages and/or to a required size based on the age of the patient, the procedure undertaken, and/or any aspect known in the art which may affect stent circumference. Accordingly, the present invention provides an external dial configured to allow a user to precisely limit the extent of crimping for a particular stent and adjust said extent of crimping on the fly as needed.

### Method of Use:

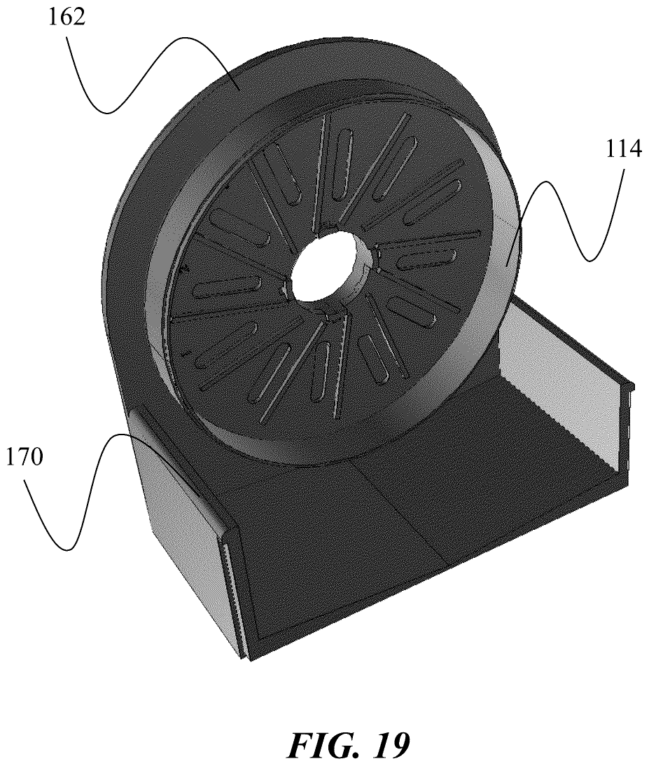

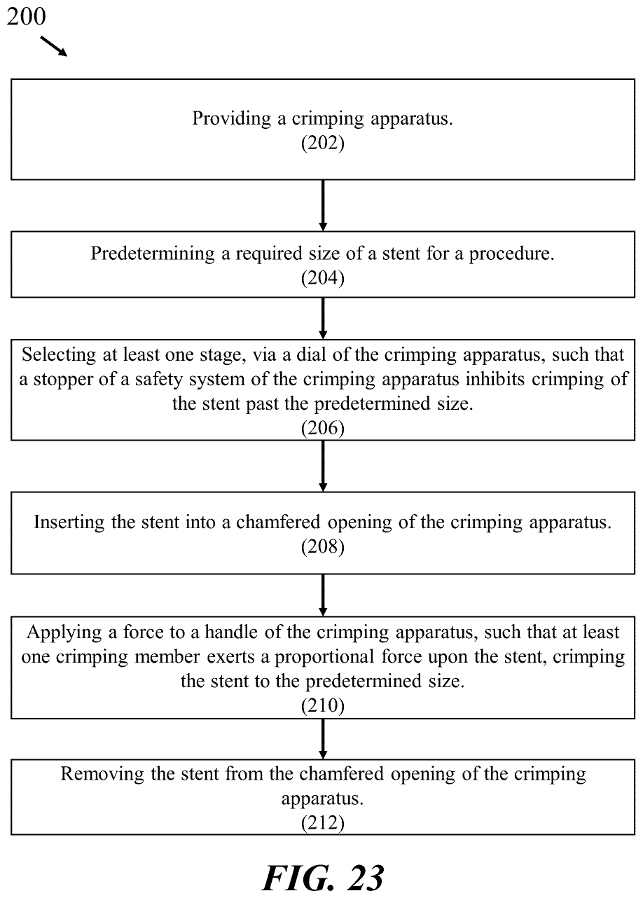

FIG. 23, in conjunction with FIGS. 1-22, depicts a flow chart providing the steps of a method 200 of crimping at least one stent 174 using a crimping apparatus 100, comprising a handle 112, a casing 176, a safety system 142, and at least one crimping member 150, according to an embodiment of the present disclosure. The steps delineated in FIG. 23 are merely exemplary of an order of crimping at least one stent 174 using crimping apparatus 100. The steps may be carried out in another order, with or without additional steps included therein.

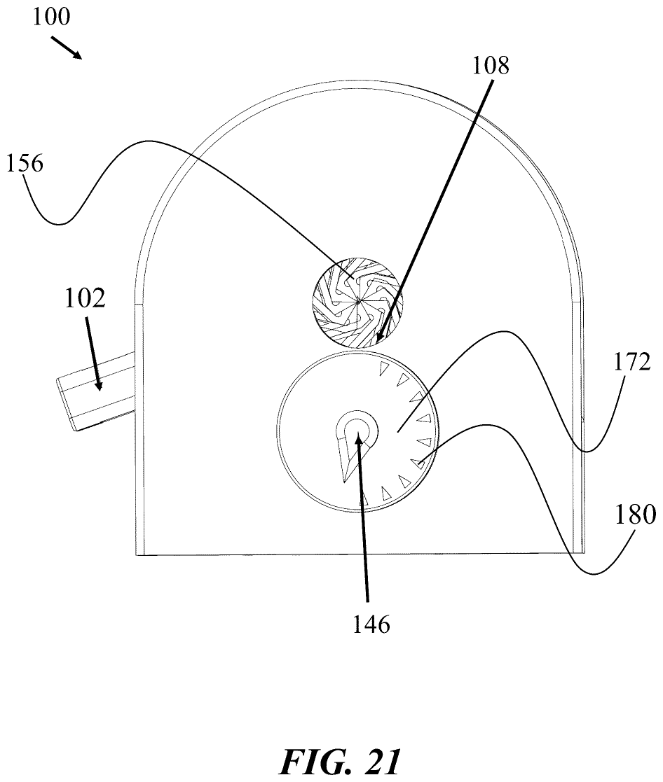

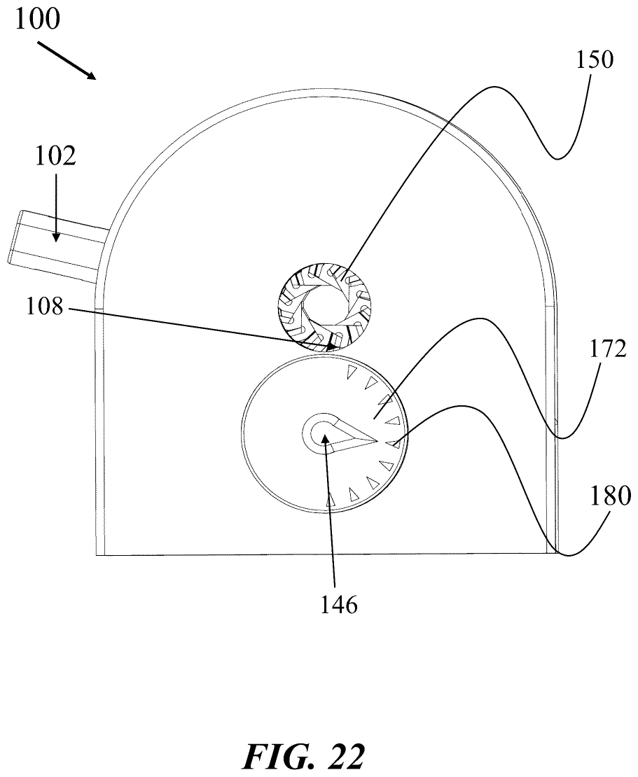

As shown in FIG. 23, in conjunction with FIGS. 1-22, in an embodiment, the method 200 begins at step 202, in which a crimping apparatus comprising a handle 112, a casing 176, a safety system 142, and at least one crimping member 150 is provided. The next step, step 204, comprises determining a required size for at least one stent 174 based on the procedure, age of the patient, health of the patient, and/or any aspect known in the art which may affect stent size. Subsequently, as shown in FIG. 22, at step 206, a predetermined size is selected via at least one stage 180 of dial 172 of safety system 142, such that at least one stopper 136 inhibits crimping of the at least one stent past the preselected size. Additionally, in an embodiment, as shown in FIGS. 20-21, safety system 142 may be disengaged such that handle 112 may be configured to rotationally translate until reaching handle stop 170. Moreover, in this embodiment, spring plunger 140 of at least one stopper 136 may be configured to be retracted, such that at least one crimping member 150 may be configured to fully decrease the circumference of the at least one aperture extending throughout crimping apparatus 100 to a negligible circumference, as shown in FIG. 21. Furthermore, at step 208, the at least one stent 174 may be inserted through opening 166 comprising a chamfered entrance 178, such that the at least one stent 174 may be disposed through crimping apparatus 100, such that each portion requiring crimping may receive a proportional force, via the at least one crimping member 150 of crimping apparatus 100. Next, at step 210 at least one user may apply a force to interface 102 of handle 112 such that as handle 112 rotationally translates toward handle stop 170, at least one crimping member 150 may be configured to exert a proportional force upon the at least one stent 174 to crimp stent 174 to the predetermined size. Finally, at step 212, the at least one stent 174 is removed from crimping apparatus 100, via opening 166 comprising chamfered entrance 178, allowing ease-of-access to the at least one crimped stent 174 such that the at least one user may retrieve the at least one crimped stent 174 without using excess force and/or comprising the integrity of the at least one crimped stent 174. Additionally, in some embodiments, once crimped stent 174 is removed, at least one alternative stent 174 may be added, as needed, such that the process may occur in real-time in a sterile room, during the procedure. Additionally, in some embodiments, a plurality of stents 174 may be added to the crimping apparatus 100, such that each of the plurality of stents 174 may be proportionally crimped by at least one crimping member 150, such that each of the plurality of stents 174 may be crimped to the predetermined size, simultaneously.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

It is also to be understood that the following claims are intended to cover all of the generic and specific features of the invention herein described, and all statements of the scope of the invention which, as a matter of language, might be said to fall therebetween.

What is claimed is:

1. A crimping apparatus for crimping a stent, the crimping apparatus comprising:
a casing, the casing having an opening for receiving the stent;
a handle rotatably mounted within the casing, wherein the handle includes a plurality of stopper holes;
a radial housing mounted within the casing in a non-rotatable manner;
at least one crimping member, wherein the at least one crimping member is translate relative to the radial housing;
a safety system, the safety system comprising a safety stopper and an external dial, wherein the external dial has a plurality of predetermined stages, wherein each stage corresponds to a particular location of the safety stopper;
the safety stopper configured to engage a stopper hole when the external dial is located at one of the plurality of predetermined stages and the handle is rotated a predetermined distance from a starting position, such that the handle is prevented from rotating further;
wherein, responsive to rotation of the handle from the starting position, the at least one crimping member reduces an effective diameter of the opening in the casing.

2. The crimping apparatus of claim 1, wherein the plurality of stopper holes in the handle are collectively arranged in an arcuate shape.

3. The crimping apparatus of claim 2, wherein the arcuate shape partially forms a Fibonacci spiral.

4. The crimping apparatus of claim 1, wherein the safety stopper includes a spring-loaded plunger.

5. The crimping apparatus of claim 1, further including an arcuate track in the casing and the safety stopper extending through the track in the casing.

6. The crimping apparatus of claim 1, wherein each stopper hole in the plurality of stopper holes includes a sloped entrance section, thereby allowing the handle to be rotated back to the starting position.

7. The crimping apparatus of claim 1, further including:
a cylindrical extension projecting outwardly from the casing;
a plurality of stage holes circumferentially spaced about the cylindrical extension;
the safety system having a stage stopper, wherein the stage stopper is configured to engage one of the plurality of stage holes when the stage stopper is aligned with one of the plurality of stage holes;
wherein the external dial location determines a location of the stage stopper relative to the plurality of stage holes.

8. The crimping apparatus of claim 1, wherein the stage stopper includes a spring-loaded plunger.

9. The crimping apparatus of claim 1, wherein the opening in the casing has a tapered entrance.

10. The crimping apparatus of claim 1, wherein the external dial includes indicia for each of the predetermined stages, wherein the indicia indicate an expected crimped diameter when the external dial of the safety system is set to one of the predetermined stages.

11. A crimping apparatus for crimping a stent to a predetermined size, the crimping apparatus comprising:
a casing, the casing having an opening for receiving the stent;
a handle rotatably mounted within the casing, wherein the handle includes a plurality of stopper holes;
a radial housing mounted within the casing in a non-rotatable manner;
at least one crimping member, wherein the at least one crimping member is configured to translate relative to the radial housing;
a safety system, the safety system comprising:
a safety stopper and an external dial;
the external dial having a plurality of predetermined stages, wherein each stage corresponds to a particular location of the safety stopper;
the external dial further including indicia for each of the plurality of predetermined stages, wherein the indicia indicate an expected crimped diameter when the external dial of the safety system is set to one of the predetermined stages;
the safety stopper configured to engage a stopper hole when the external dial is located at one of the plurality of predetermined stages and the handle is rotated a predetermined distance from a starting position, such that the handle is prevented from rotating further;
wherein, responsive to rotation of the handle from the starting position, the at least one crimping member reduces an effective diameter of the opening in the casing.

12. The crimping apparatus of claim 11, wherein the plurality of stopper holes in the handle are collectively arranged in an arcuate shape.

13. The crimping apparatus of claim 12, wherein the arcuate shape at least partially approximates a Fibonacci spiral.

14. The crimping apparatus of claim 11, wherein the safety stopper includes a spring-loaded plunger.

15. The crimping apparatus of claim 11, further including an arcuate track in the casing, wherein the safety stopper is configured to extend through the track in the casing.

16. The crimping apparatus of claim 11, wherein each stopper hole in the plurality of stopper holes includes a sloped entrance section, thereby allowing the handle to be rotated back to the starting position.

17. The crimping apparatus of claim 11, further including:
a cylindrical extension projecting outwardly from the casing;
a plurality of stage holes circumferentially spaced about the cylindrical extension;
the safety system having a stage stopper, wherein the stage stopper is configured to engage one of the plurality of stage holes when the stage stopper is aligned with one of the plurality of stage holes;
wherein the external dial location determines a location of the stage stopper relative to the plurality of stage holes.

18. The crimping apparatus of claim 11, wherein the stage stopper includes a spring-loaded plunger.

19. The crimping apparatus of claim 11, wherein the opening in the casing has a tapered entrance.

20. A method of crimping a stent, comprising:
acquiring a crimping apparatus for crimping a stent to a predetermined size, the crimping apparatus comprising:
a casing, the casing having an opening for receiving the stent;
a handle rotatably mounted within the casing, wherein the handle includes a plurality of stopper holes;
at least one crimping member, wherein the at least one crimping member is configured to reduce a diameter of the opening;
a safety system, the safety system comprising a safety stopper and an external dial, wherein the external dial having a plurality of predetermined stages, wherein each stage corresponds to a particular location of the safety stopper;
the safety stopper configured to engage a predetermined stopper hole when the external dial is located at one of the plurality of predetermined stages and the handle is rotated a predetermined distance from a starting position, such that the handle is prevented from rotating further;
wherein, responsive to rotation of the handle from the starting position, the at least one crimping member reduces the diameter of the opening in the casing;
inserting the stent into the opening;
setting the external dial to one of the plurality of predetermined stages corresponding to an expected crimped diameter;
rotating the handle from the starting position until the safety stopper engages one of the predetermined stopper holes; and
rotating the handle back to the starting position after the handle stops rotating as a result of the safety stopper engaging one of the predetermined stopper holes.
